(19) United States
(12) Patent Application Publication
(10) Pub. No.: US 2005/0271648 A1
(43) Pub. Date: Dec. 8, 2005

(54) Carbon nanotube structure and production method thereof

(75) Inventor: Yukihiro Sugiyama, Osaka (JP)

Correspondence Address: MCDERMOTT WILL & EMERY LLP, 600 13TH STREET, N.W., WASHINGTON, DC, 20005-3096, US

(73) Assignee: Sanyo Electric Co., Ltd., Osaka (JP)

(21) Appl. No.: 11/123,170

(22) Filed: May 6, 2005

Related U.S. Application Data
(63) Continuation of application No. PCT/JP03/14177, filed on Nov. 7, 2003.

(30) Foreign Application Priority Data
Nov. 7, 2002 (JP) 2002-324373

(51) Int. Cl. A61K 38/17; A61K 33/44

(52) U.S. Cl. 424/125; 424/488; 514/2

(57) ABSTRACT

Modified molecules (119) and carbon nanotubes (105) are dispersed in a dispersion medium (121). The resulting dispersion solution is spread over the surface of a subphase (125) in a Langmuir trough (113) to obtain carbon nanotube structures (131) comprising the carbon nanotubes (105) and the modified molecules (129) covering the sidewall of the carbon nanotubes (105).

This is a continuation of International Application PCT/JP2003/014177, filed Nov. 7, 2003.

### TECHNICAL FIELD

The present invention relates to carbon nanotube structures and a method for producing carbon nanotube structures.

### BACKGROUND ART

Recently carbon nanotubes have attracted a great interest as a carbon material with nanoscale microstructure that essentially is a rolled graphite sheet forming a cylindrical structure. Since the sidewall of a carbon nanotube is made up with stable carbon hexagons, carbon nanotubes are generally inert to chemical reaction. Thus, attempts have been made to alter the surface properties of carbon nanotubes: the carbon nanotubes were coated on the sidewall or were cut into short segments to increase its solubility (Non-Patent Article 1).

Despite these efforts, a significant demand still exists for novel surface treatment technologies that provide carbon nanotubes with new surface properties and thereby broaden its applications.

Non-Patent Article 1: Kazuyoshi Tanaka ed., “Frontiers in Chemistry 2—A challenge to develop carbon nanotubes and nanodevices” 1st ed., Kagaku-Dojin Co., Ltd., Jan. 30, 2001, pp. 100-102.

### DISCLOSURE OF THE INVENTION

In view of the above-described state of the art, an objective of the present invention is to provide a novel surface treatment technology for carbon nanotubes.

According to the present invention, there is provided a carbon nanotube structure that comprises a carbon nanotube and a layer comprising a polymer that covers the sidewall of the carbon nanotube.

Having its sidewall covered with the layer comprising the polymer, the carbon nanotube structure according to the present invention makes it possible to impart new surface properties to carbon nanotubes and, thus, broaden its applications.

As used herein, the phrase “to cover the side wall of carbon nanotubes” means to cover an area of the side wall of carbon nanotubes. What the phrase is intended to mean includes an embodiment in which polymer molecules are wound about a carbon nanotube to cover an area of its sidewall. The phrase also includes an embodiment in which polymer molecules form a layer to cover the sidewall, i.e., form a polymer covering layer. The term “covering layer” as used herein means a densely formed covering layer that extends over an area of the sidewall of carbon nanotubes.

Thus, the term “cover(ing)” as used herein encompasses both polymer molecules wound about the nanotube and polymer molecules forming a layer about its side wall.

In one embodiment of the carbon nanotube structure of the present invention, the polymer molecules may directly cover the sidewall of carbon nanotubes. This ensures the firm covering of the sidewall of carbon nanotubes.

The area of carbon nanotubes to be covered may be a part of its sidewall or entire sidewall.

According to the present invention, there is also provided a carbon nanotube structure that includes a carbon nanotube and a polymer wound about the carbon nanotube.

In the carbon nanotube structure according to the present invention, the polymer is wound about the carbon nanotube at its sidewall. This structure can stably hold the polymer covering over the surface of the carbon nanotube. The wound polymer ensures the firm covering of individual carbon nanotubes and thus makes it possible to alter its surface properties. As used herein, the term “polymer” or “polymer molecule” refers to a molecule that has a backbone long enough to be wound about the carbon nanotube. The phrase “the polymer is wound about the carbon nanotube” as used herein means that the polymer chain extends over the circumference of the carbon nanotube to cover its surface.

According to the present invention, there is provided a method for producing a carbon nanotube structure. This method involves dispersing carbon nanotubes and polymer molecules in a dispersion medium to form a dispersion solution, and spreading the dispersion solution on the surface of a liquid to allow the polymer molecules to wind about the carbon nanotubes.

This simple method for winding the polymer molecules about the carbon nanotubes enables stable and effective production of the carbon nanotube structure of the present invention.

In the carbon nanotube structure of the present invention, the aforementioned layer may be formed to uniformly cover the entire sidewall of the carbon nanotube. This construction further improves the dispersion stability of the carbon nanotubes.

In the carbon nanotube structure of the present invention, the polymer may form a uniform layer to cover the sidewall of the carbon nanotube. This construction reduces the deviation of the surface properties of the carbon nanotube structure.

In the carbon nanotube structure of the present invention, the thickness of the foregoing layer may be in the range from 1 nm to 100 nm.

In the carbon nanotube structure of the present invention, the polymer may be an insulator. In this manner, an insulative layer can be formed over the sidewall of the carbon nanotube.

In the carbon nanotube structure of the present invention, the polymer may be a biological polymer. In this manner, new surface properties can be imparted to the carbon nanotube.

In the carbon nanotube structure of the present invention, the polymer may be insoluble in water. In this manner, exfoliation of the polymer layer and penetration of water molecules into the carbon nanotube surface can be prevented and, thus, the carbon nanotube structure can be stabilized in water.

In the carbon nanotube structure of the present invention, the polymer may include a polypeptide. The polymer for use in the carbon nanotube of the present invention may be a polypeptide. The backbone of a polypeptide enables the covering polymer to stably cover the carbon nanotube. In addition, the properties of the amino acid side chain residues can be utilized to impart different surface properties to the sidewall of the carbon nanotube.

In the carbon nanotube structure of the present invention, the polymer may be a denatured protein.

In one method for producing the carbon nanotube structure of the present invention, a protein may be used as the polymer. In such a case, the aforementioned dispersion solution may be spread on the surface of a liquid to denature the protein, which then is applied to the sidewall of the carbon nanotube to be covered therewith. In this manner, the formed polymer layer may be further stabilized.

Unlike normal protein, the denatured protein generally has its hydrophobic domains exposed, which facilitates covering of the polymer layer to the sidewall of the carbon nanotube. By allowing the protein-dispersed solution to spread over the surface of a certain liquid, the protein can be effectively denatured with the help of the surface tension at the gas/liquid interface, exposing its hydrophobic domains. The term “denature” of the protein as used herein refers to disruption of the conformation of a protein molecule and associated loss of its function. The term includes any conformational change other than cleavage of the primary structure, or the amino acid sequence, of the protein molecule and encompasses any degree of conformational change.

In the present invention, the polymer may be a membrane protein.

Membrane proteins generally contain hydrophobic regions, which facilitate cohesion of the polymer layer to the sidewall of the carbon nanotube and help stabilize the polymer layer.

According to the present invention, there is provided a method for solubilizing carbon nanotubes. This method involves adding carbon nanotubes to a dispersion medium containing a membrane protein. Since the method uses membrane proteins, carbon nanotubes can be solubilized in a stable manner.

When the dispersion medium containing the membrane protein is used in the solubilizing method of the present invention, the dispersion medium may be such that it does not cause the intramolecular hydrophobic region of the membrane protein to be exposed on the outside of the molecule. In this manner, the carbon nanotubes can be stably solubilized.

According to the present invention, there is provided a method for preserving carbon nanotubes. This method involves maintaining carbon nanotubes in a liquid containing a membrane protein. In the preserving method according to the present invention, the membrane protein present in the liquid serves to keep carbon nanotubes well-dispersed in the medium.

Furthermore, the re-dispersibility of the carbon nanotubes can be ensured. In the preserving method of the present invention, a liquid may be used that does not cause the intramolecular hydrophobic region of the membrane protein to be exposed on the outside of the molecule. The use of such a liquid allows the carbon nanotubes to be preserved in a highly dispersed state.

As described above, the present invention provides a novel surface treatment technology for carbon nanotubes that involves covering the sidewall of carbon nanotubes with a polymer layer.

### BRIEF DESCRIPTION OF THE DRAWINGS

These and other objectives as well as novel features and advantages of the present invention will become apparent from the following description of preferred embodiments when considered in conjunction with the accompanying drawings, in which:

FIG. 1 is a diagram illustrating a method for producing carbon nanotube structures according to an embodiment of the present invention;

FIGS. 2A and 2B are diagrams showing one exemplary construction of a transistor according to an embodiment of the present invention;

FIGS. 3A, 3B, 3C, and 3D are illustrative diagrams showing a method for producing the transistor according to the embodiment of the present invention;

FIGS. 4A, 4B, 4C, and 4D are illustrative diagrams showing a method for producing the transistor according to the embodiment of the present invention;

FIGS. 5A, 5B, and 5C are illustrative diagrams showing a method for producing the transistor according to the embodiment of the present invention;

FIG. 6 is an illustrative diagram showing a method for producing a carbon nanotube structure according to an embodiment of the present invention;

FIGS. 7A and 7B are each an AFM image of a single-walled carbon nanotube structure according to one example;

FIGS. 8A, 8B, 8C, 8D, and 8E are illustrative diagrams showing a production sequence of the carbon nanotube structure according to the embodiment of the present invention;

FIG. 9 is an AFM image of the single-walled carbon nanotube structure according to one example;

FIG. 10 is a TEM image of the single-walled carbon nanotube structure according to one example;

FIG. 11 is an AFM image of a multi-walled carbon nanotube structure according to one example; and

FIG. 12 is a TEM image of the multi-walled carbon nanotube structure according to one example.

### BEST MODE FOR CARRYING OUT THE INVENTION

The present invention will now be described in detail with reference to the accompanying drawings throughout which like elements are assigned like numerals and their description will not be repeated.

### First Embodiment

A carbon nanotube structure according to the present embodiment includes a covering of modified molecules extending over its sidewall. The covering may be formed over a particular area of the carbon nanotube surface or over the entire sidewall of the carbon nanotube. The modified molecules for use in this embodiment are polymers. The covering may be a densely formed layer extending over an area of the sidewall of the carbon nanotube.

In this embodiment, the modified molecule may be wound about the carbon nanotube to cover the sidewall. The term “layer” as used herein applies also to the polymers wound under certain conditions to form a covering.

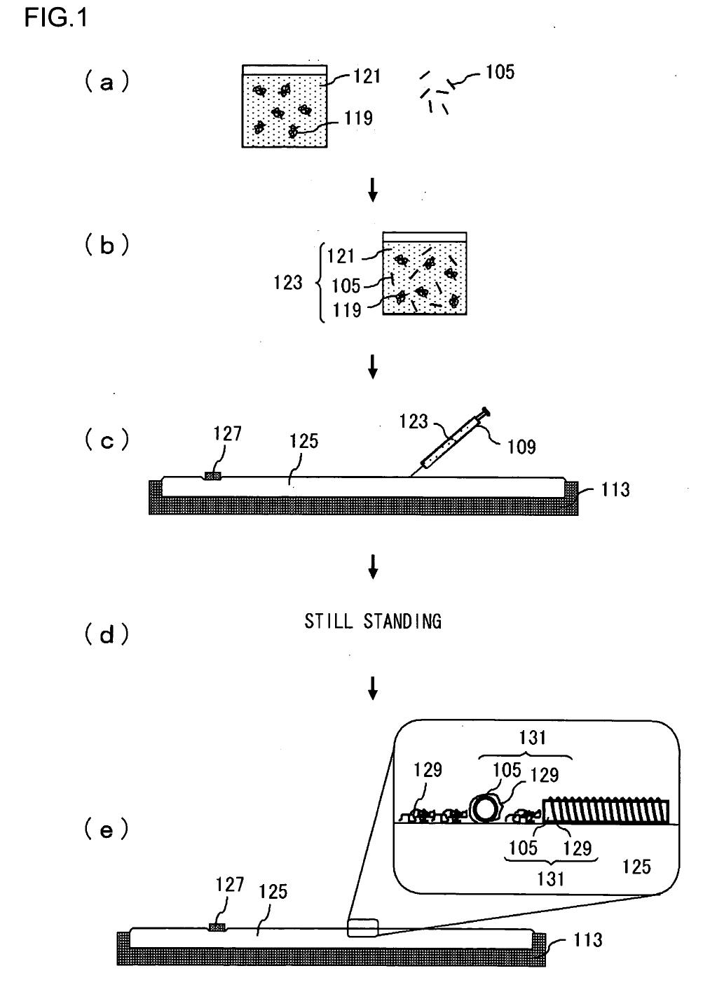

A description will now be given of one example in which a modified molecule is wound about a carbon nanotube to cover the sidewall of the nanotube. The carbon nanotube structure in this embodiment has a construction in which the modified molecule is wound about the carbon nanotube to cover its sidewall. FIG. 1 shows one exemplary production method of a carbon nanotube structure 131.

Referring now to FIG. 1, modified molecules 119 are first dispersed in a dispersion medium 121 (FIG. 1(a)). Carbon nanotubes 105 are then added and dispersed to obtain a dispersion solution 123 (FIG. 1(b)). A sonicator or the like may be used to disperse these components.

Using a syringe 109 or the like, the dispersion solution 123 is spread over the surface of a subphase 125 placed in a reservoir (FIG. 1(c)). As an example of the reservoir, FIG. 1 shows a Langmuir trough 113 equipped with a movable barrier 127.

Once spread, the dispersion solution 123 is allowed to stand so that the modified molecules 119 can undergo conformational change due to the interfacial tension and become wound about the carbon nanotubes 105, covering the sidewalls of the nanotubes 105 (FIG. 1(d)). This results in a carbon nanotube structure 131 consisting of the carbon nanotube 105 and the modified molecule 129 wound about the carbon nanotube 105 and having had its conformation changed.

In FIG. 1, the carbon nanotubes 105 may be any of single-walled carbon nanotubes (SWCNTs), multi-walled carbon nanotubes (MWCNTs) and carbon nanohorns (CHNs). The carbon nanotubes 105 may have any diameter and length; for example, they may be from 0.3 nm to 100 nm in diameter and from 50 nm to 10 μm in length.

The modified molecules 119 may be any type of molecules that can be wound about the carbon nanotubes 105 and modify the surface of the carbon nanotubes 105, including various synthetic polymers and biological polymers. Preferably, the modified molecule is a molecule that can be wound about the carbon nanotube to form a layer to cover the sidewall of the carbon nanotube. For the modified molecule 119 to be wound about the carbon nanotube 115, it is preferably a synthetic polymer having a hydrophobic backbone. Examples of such synthetic polymers include polyolefins and polyamides. Proteins, DNA, and other biological molecules may also be used as the modified molecule 119, as may polymers insoluble in water.

When a protein is used as the modified molecule 119, the protein is preferably such that it contains a hydrophobic region that is hidden within the hydrophilic region when the protein is in the dispersion medium 121 but is exposed on the outside at the gas/liquid interface once the dispersion solution is spread over the subphase 125. It is preferred that when the hydrophobic region is exposed, the protein is hydrophobisized to a relatively high degree. Such a protein can be wound about the carbon nanotube 105 to form a covering over the surface of the carbon nanotube 105 as it is denatured at the gas/liquid interface. In this manner, the carbon nanotube structures 131 can be obtained in a stable manner. The denatured protein used to cover the sidewall of the carbon nanotube 105 makes it possible to provide the covering in the form of a layer and make a thin covering layer.

A variety of membrane proteins, such as bacteriorhodopsins, can also be used as the modified molecule 119. Membrane proteins are generally water-insoluble and are rich in hydrophobic amino acids. For this reason, they can be effectively wound about the carbon nanotubes 105 to form a dense covering layer over the surface of the carbon nanotubes 105. The backbone length of the modified molecule 119 is properly selected depending on the length of the carbon nanotube 105 or the desired application of the carbon nanotube structure 131.

The dispersion medium 121 is properly selected from organic solvents in which the modified molecules 119 can be dispersed in a relatively stable manner, and mixtures or aqueous solutions of such organic solvents. Also, the subphase 125 is properly selected depending on the type of the dispersion medium 121 or the modified molecules 119. For example, when a bacteriorhodopsin is used to serve as the modified molecule 119, an aqueous solution of an organic solvent can be used as the dispersion medium 121. Specifically, an aqueous solution of DMF (dimethylformamide) or DMSO (dimethylsulfoxide) can be used as the dispersion medium 121. The subphase 125 may be an acidic aqueous solution having a pH value in the range of 2 to 6, and preferably in the range of 3 to 4. In this manner, a uniform composite monolayer consisting of the carbon nanotube 105 and bacteriorhodopsin can be formed over the subphase. The production method of carbon nanotube structure 131 that involves bacteriorhodopsin will be described in further detail in Examples below.

By using as the modified molecule 119 a polymer that includes a hydrophobic backbone and a hydrophilic side-chain group such as hydroxyl and carboxyl, the dispersibility of the carbon nanotube 105 in water can be significantly increased. The modified molecule 129 wound about the carbon nanotube 105 can uniformly cover the sidewall of the carbon nanotube 105. By changing the side chain of the modified molecule 119, the dispersibility of the carbon nanotubes 105 in different solvents can be adjusted as desired.

According to this embodiment, a dense covering layer can be formed at least over an area of the surface of the carbon nanotube 105. This makes it possible to impart a new surface property to the carbon nanotube. For example, the carbon nanotube structure 131 that can remain dispersed in water in a stable manner can be obtained. Furthermore, by using an insulative material as the modified molecule 119, a dense insulative layer can be formed over the surface of the carbon nanotube 105. Such carbon nanotubes 105 can effectively serve as electronic devices such as transistors and capacitors with the covering layer serving as the gate insulative film. The chemical modification of the modified molecule 119 can also be utilized.

In this method, the covering layer of the modified molecule 129 formed over the surface of the carbon nanotube 105 can be made to have a uniform thickness by using multi-walled carbon nanotubes as the carbon nanotube 105. When single-walled carbon nanotubes are used, the wound layer can be made to have a predetermined pitch.

The construction of the carbon nanotube structure 131 in which the modified molecule 129 is wound about the carbon nanotube 105 allows the modified molecule 129 to be stably attached to the sidewall of the carbon nanotube 105. This improves the dispersion stability and the storage stability of the carbon nanotube structure 131. In addition, the insulation of the sidewall of the carbon nanotube 105 can be increased by using an insulative material as the modified molecule 119.

In the carbon nanotube structure 131 having the modified molecule 129 wound thereabout, the covering may be formed at a predetermined pitch on the sidewall of the carbon nanotube. The thickness of this covering can be properly controlled during formation of the covering. For example, the electrical property of the surface of the carbon nanotube 105 can be varied by controlling the covering thickness in the range of 1 nm to 100 nm. When the modified molecule 129 is wound about the carbon nanotube 105 to form the covering layer, the layer may be either single-layered or multi-layered.

Alternatively, the carbon nanotube structure 131 according to the first embodiment may consist of the carbon nanotube 105 and a uniform covering layer formed over the sidewall of the carbon nanotube 105.

In such a carbon nanotube structure 131, the covering layer may be 0.1 nm thick or more, and preferably 1 nm thick. In this manner, the surface property of the carbon nanotube 105 can be varied as desired. The covering layer may be 10 nm thick or less, and preferably 5 nm thick or less. In this manner, the covering layer can be formed as a thin film. This further increases the dispersion stability of the carbon nanotube 105 while fully exploiting its characteristics. Such a thin film can be efficiently formed over the carbon nanotube 105 using minimum amounts of the modified molecule 119. By using an insulative material as the modified molecule 119, a thin insulative film can be formed over the sidewall of the carbon nanotube 115. Such an insulative film can serve as a tunnel layer and makes the nanotube 105 suitable for use as various electronic devices.

### Second Embodiment

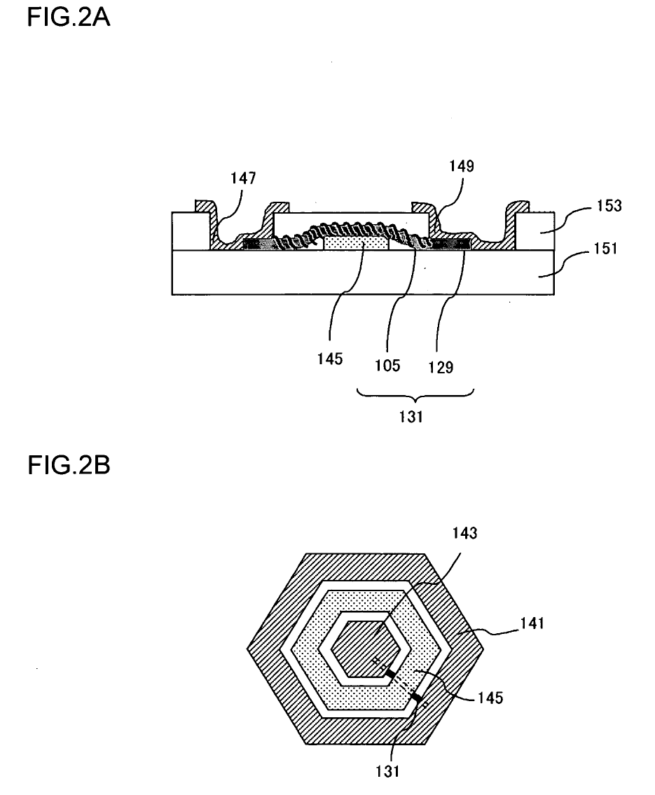

A second embodiment of the present invention concerns a transistor that uses the carbon nanotube structure as its wirings. FIGS. 2A and 2B each show one exemplary construction of such a transistor with FIG. 2A being a cross-sectional view and FIG. 2B a plan view. Referring to FIG. 2A, the transistor includes a source electrode 147 and a drain electrode 149 that are connected via a carbon nanotube structure 131. The carbon nanotube structure 131 includes a modified molecule 129 to serve as an insulative film between the carbon nanotube structure and a gate electrode 145. As shown in FIG. 2B, the transistor has a first electrode 141 and a second electrode 143. The first electrode 141 is arranged such that it is spaced apart from and encircles the second electrode 143. One of the first electrode 141 and the second electrode 143 is assigned as the source electrode 147 and the other as the drain electrode 149. This arrangement of the electrodes allows the source electrode 147 and the drain electrode 149 to be connected via the carbon nanotube structure 131 relatively easily, leading to an increase in the productivity of the transistor.

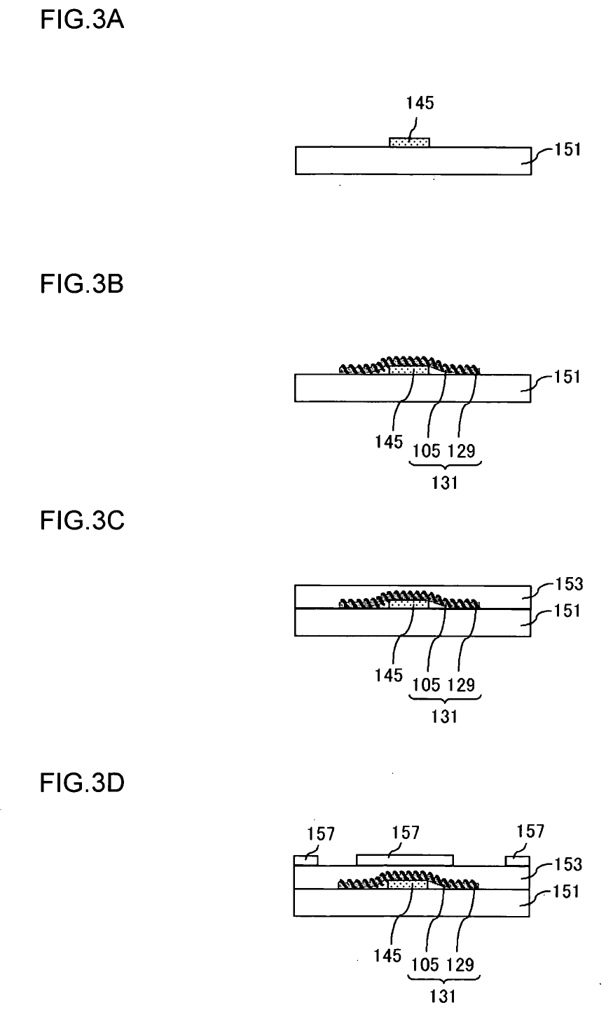

One production method of the transistor of FIG. 2 will now be described with reference to FIG. 3A through FIG. 5C. A metal layer to serve as the gate electrode 145 is first deposited on the surface of a substrate 151 (FIG. 3A). The metal layer to serve as the gate electrode 145 may be formed of metals such as Al, Cu, Ag, Au, Pt, Ti, Co, and Pd, or alloys thereof. The gate electrode 145 can be formed by vapor deposition or sputtering.

Next, the carbon nanotube structure 131 to serve as the channel is disposed on the surface of the gate electrode 145 (FIG. 3B). The carbon nanotube structure 131 is disposed on the surface of the gate electrode 145 in the following manner.

In the same manner as in the first embodiment, the carbon nanotube structures 131 spread over the surface of a subphase 125 are prepared (FIG. 1(e)). A protein such as bacteriorhodopsin is used as the modified molecule 119. Alternatively, a purple membrane containing a bacteriorhodopsin as its protein component may be used.

Once spread over the surface of the subphase 125, the carbon nanotube structures 131 are allowed to adhere to the surface of the substrate 151 having the electrode 145 formed thereon. This is done by using the horizontal lifting method. In the horizontal lifting method, the substrate 151 is held horizontally above the liquid surface and is brought into and out of contact with the liquid surface to cause the carbon nanotube structures 131 spread over the liquid surface to adhere to the surface of the substrate 151 with the gate electrode 145 formed thereon.

Thus, the carbon nanotube structures 131 have been disposed on the surface of the substrate 151 with the gate electrode 145 formed thereon.

A mask 153 is then deposited on the surface of the oriented film of the carbon nanotube structures 131 by using techniques such as plasma CVD (FIG. 3C). The mask 153 may be formed of, for example, SiO₂. The mask is formed to have a thickness of, for example, 1 nm to 1 μm thick.

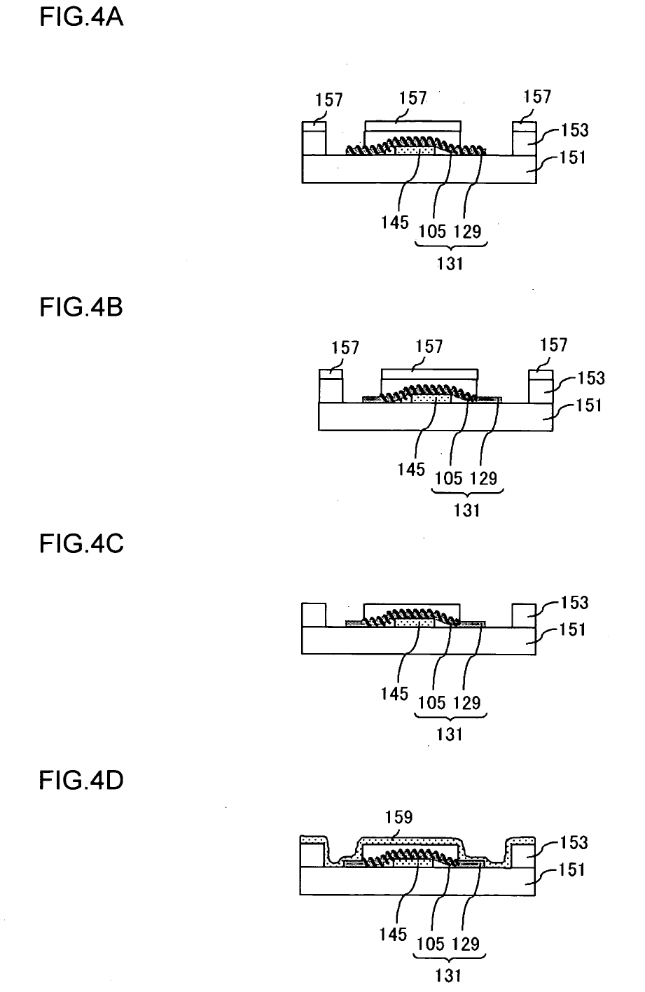

A resist film 157 is then formed for removing the mask 153 in the areas corresponding to the source electrode 147 and the drain electrode 149 (FIG. 3D). The mask is then removed by dry-etching or wet-etching where the source electrode 147 and the drain electrode 149 are to be formed (FIG. 4A). This leaves each end of the carbon nanotube structure 131 exposed. At least part of the exposed modified molecule 129 on the sidewall of the carbon nanotube structure 131 is then removed by using techniques such as ashing (FIG. 4B). Subsequently, the resist film 157 is removed with a solvent that dissolves the resist film 157 but not the mask 153 (FIG. 4C).

A metal film 159 serving as the source electrode and the drain electrode is then deposited over the entire top surface of the substrate 151 (FIG. 4D). As with the gate electrode 145, the metal film 159 can be deposited by using techniques such as vapor deposition and sputtering. The metal film 159 may be formed of metals that can form carbides, such as Ti and Cr; low resistance metals, such as Au, Pt, and Cu; or alloys thereof, such as Au—Cr alloy. Of these, the metals that can form carbides are particularly preferred since they can reduce the contact resistance between the metal film 159 and the carbon nanotube 105. Au, a precious metal, is also preferred due to its low specific resistance.

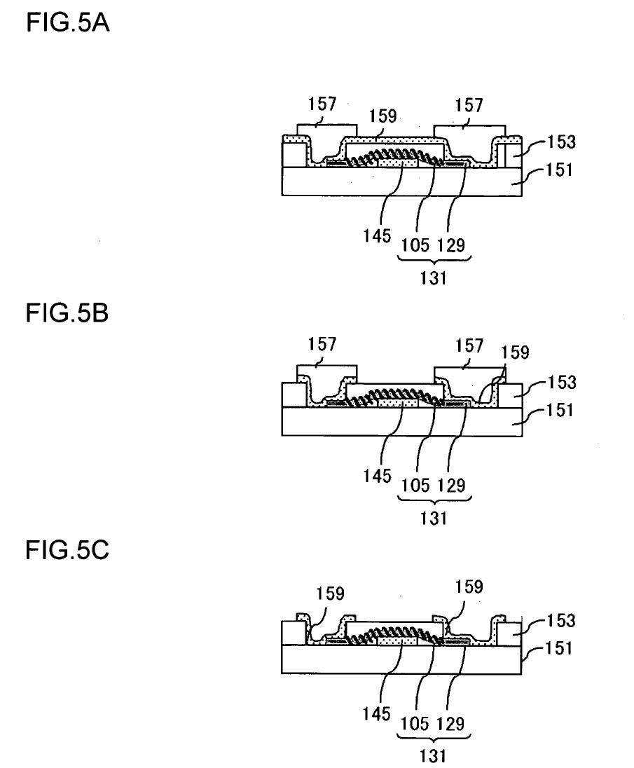

This is followed by the formation of a resist film 157 on the metal film 159 (FIG. 5A). The resist film 157 is used as a mask to etch a pattern on the metal film 159 (FIG. 5B) and is later removed to expose the metal film 159. One of the two metal film areas 159 is assigned as the source electrode 147 and the other as the drain electrode 149 (FIG. 5C).

Thus, a transistor having a construction shown in FIG. 2 has been obtained in which the carbon nanotube 105 exposed at either end of the carbon nanotube structure 131 is electrically connected to the source electrode 147 and the drain electrode 149. By using the carbon nanotube structure 131, the modified molecule 129 can serve as an insulative film that provides insulation between the gate electrode 145 and the carbon nanotube 105. Thus, the use of the carbon nanotube structure 131 eliminates the need to provide a thin insulative film between the gate electrode 145 and the carbon nanotube 105 after formation of the gate electrode 145 and can thus increase the productivity. In addition, the modified molecule 129 wound about the carbon nanotube 105 enables uniform formation of the insulative film on the sidewall of the carbon nanotube. 105. The use of the carbon nanotube structure 131 also allows formation of an insulative film with uniform thickness, leading to an increased reliability.

The production process of the carbon nanotube structures 131 may include, following the step depicted in FIG. 1(e), an additional step of moving the movable barrier 127 of the Langmuir trough 113 serving as a partition to compress the spread carbon nanotube structures 131. By compressing the spread carbon nanotube structures 131, the carbon nanotube structures 131 can be oriented. For example, when the bacteriorhodopsin present in the purple membrane is used as the modified molecule 119, the compression is performed at a rate of 20 cm²/min until the surface pressure reaches 15 mN/m. The orientation of the carbon nanotube 105 can be confirmed by, for example, AFM. The modified molecules 129, or denatured protein molecules, that do not become wound about the carbon nanotubes 105 uniformly cover the subphase 125, serving as a filling material. This material serves as a support for the carbon nanotubes 105 and helps maintain the orientation of the carbon nanotubes 105.

### Third Embodiment

A third embodiment of the present invention concerns a method for solubilizing membrane proteins and analyzing their primary structures.

The membrane protein to be analyzed may be, for example, a bacteriorhodopsin. To conduct analysis, a carbon nanotube structure 131 is prepared in the manner as depicted in FIG. 1. In the step shown in FIG. 1(a), a purple membrane containing bacteriorhodopsin is dispersed in the dispersion medium 121.

Surfactants have been traditionally used to solubilize membrane proteins. In this embodiment, the carbon nanotube structure 131 makes it possible to collect a membrane protein from a cell membrane one molecule after another.

In addition, the modified molecule 129 wound about the carbon nanotube 105 facilitates fragmentation of the membrane proteins by enzymes or other reagents. After fragmentation of the membrane proteins, the remaining macromolecules such as the carbon nanotubes 105 can be easily removed by techniques such as centrifugation and ultrafiltration. Also, the carbon nanotube structure 131 may be used as a probe for AFM.

### Fourth Embodiment

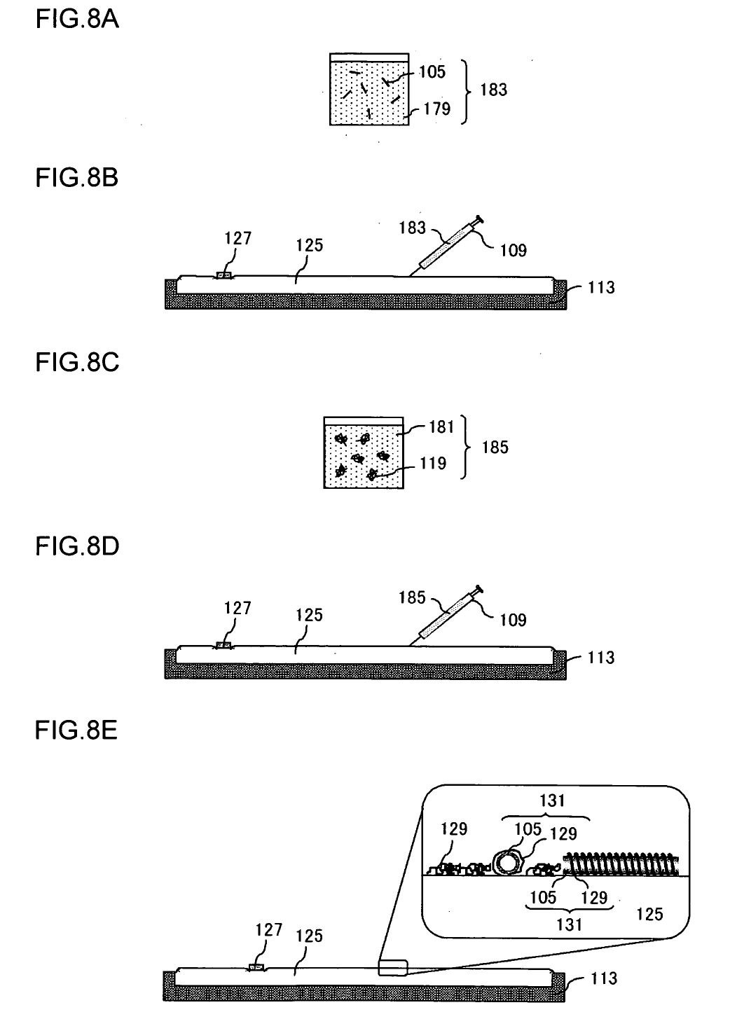

A forth embodiment of the present invention concerns another production method of the carbon nanotube structures. Procedures of the production of the carbon nanotube structures are depicted in FIGS. 8A through 8E. The method previously described with reference to FIG. 1 can be generally applied to this embodiment as well. The difference in this embodiment is that the dispersion solution 183 of the carbon nanotubes 105 and the dispersion solution 185 of the modified molecules 119 are individually prepared and spread over the subphase 125, the dispersion solution 183 of the carbon nanotube first, then the dispersion solution 185 of the modified molecules 119.

First, the carbon nanotubes 105 are dispersed in the dispersion medium 179 to prepare the dispersion solution 183 (FIG. 8A). The dispersion medium 179 may be a 10 v/v % to 90 v/v % aqueous solution of DMF or a 10 v/v % to 90 v/v % aqueous solution of DMSO. These media allow the carbon nanotubes 105 to remain well-dispersed in the dispersion solution 183. The carbon nanotubes 105 may be dispersed by sonication. The resulting dispersion solution 183 is then spread over the subphase 125 (FIG. 8B).

Meanwhile, the modified molecules 119 are dispersed in the dispersion medium 181 to prepare the dispersion solution 185 (FIG. 8C). The dispersion medium 181 may be any organic solvent or its aqueous solution in which the modified molecules 119 can be stably dispersed. As described below, these media ensure stable formation at the gas/liquid interface of the covering of the modified molecules 129 over the sidewall of the carbon nanotubes 105 upon spreading of the dispersion liquid 185 over the subphase 125.

The dispersion medium 181 is preferably one in which the carbon nanotubes 105 can be dispersed in a relatively stable manner. Such a dispersion medium allows the modified molecules 129 to firmly cover the sidewall of the individual carbon nanotubes 105. The dispersion solution 185 may be the same solution as the one used as the dispersion medium 121 in the technique described above with reference to FIG. 1. The dispersion solution 185 may be an identical solution to the dispersion solution 183. By using the same solution for the dispersion solution 185 and the dispersion solution 183, the modified molecules 129 can firmly cover the surface of the carbon nanotubes 105 upon spreading of the dispersion solutions over the subphase 125.

Subsequent to the spreading of the dispersion solution 183, the resulting dispersion solution 185 is further spread over the subphase 125 (FIG. 8D). Upon spreading of the dispersion solution 185, the modified molecules 119 and the carbon nanotubes 105 are mixed with one another and, as a result, the modified molecules 129 cover the sidewall of the carbon nanotubes 105, forming the carbon nanotube structures 131 (FIG. 8E).

Thus, the carbon nanotube structures 131 can be stably produced even when the dispersion solution 183 of the carbon nanotubes 105 and the dispersion solution 185 of the modified molecules are separately spread over the subphase 125, one after the other.

When a multi-walled carbon nanotubes are used as the carbon nanotubes 105 for use in the method according to the fourth embodiment, the covering layer of the modified molecules 129 formed over the surface of the carbon nanotubes 105 tends to have a uniform thickness. When the single-walled carbon nanotubes are used, the resulting covering consists of the modified molecule 129 wound at a predetermined pitch, forming a layer. In the latter case, a layer consisting of the modified molecule 129 wound in a predetermined state can be obtained.

While the use of the carbon nanotube structures of the present invention has been described in the foregoing description in terms of several embodiments, they can be extended to other applications. For example, the carbon nanotube structures obtained by the method shown in FIG. 1 can be used as cold cathodes of field emission devices. When a high voltage is applied to the carbon nanotube structure 131, which includes the carbon nanotube 105 having its surface covered with the modified molecule 129, the carbon nanotube structure emits electrons in a stable manner.

Having its carbon nanotube 105 covered with the insulative modified molecule 129, the carbon nanotube structure 131 obtained by the method shown in FIG. 1 can be used as a covered wire. In addition, the carbon nanotube 105, having an electric double layer structure with a cylindrical concentric tube, can serve as a memory.

The carbon nanotube structure 131 obtained by the method shown in FIG. 1 can also be applied to nanomechanics, such as nanoforceps. This device works based on the principle that when a current flows through the carbon nanotube structure 131 placed in a magnetic field perpendicular to the current, a force acts upon the carbon nanotube structure 131.

Furthermore, the carbon nanotube structure 131 obtained by the method shown in FIG. 1 can be applied to nanostereostructure. Specifically, one of antigen and antibody is immobilized onto the modified molecule 129 on the surface of the carbon nanotube structure 131. When this carbon nanotube structure 131 is mixed in a solution with the carbon nanotube structure 131 with the modified molecule 129 having the other of the antigen and the antibody, the antigen/antibody interaction takes place to form nanostereostructure. By changing combination of antigen and antibody to combination of other type of molecules, such as biotin and avidin, that have a different number of binding sites, different nanostereostructure can be obtained. The resulting nanostereostructure can be used as nanocircuits or three-dimensional nanowirings.

It should be appreciated by those skilled in the art that the specific embodiments disclosed above have been given by way of example only and various modifications may be made to components and combination of processes in these embodiments and that such modifications are also within the scope of the invention. The present invention will now be described in further detail with reference to Examples.

### EXAMPLE

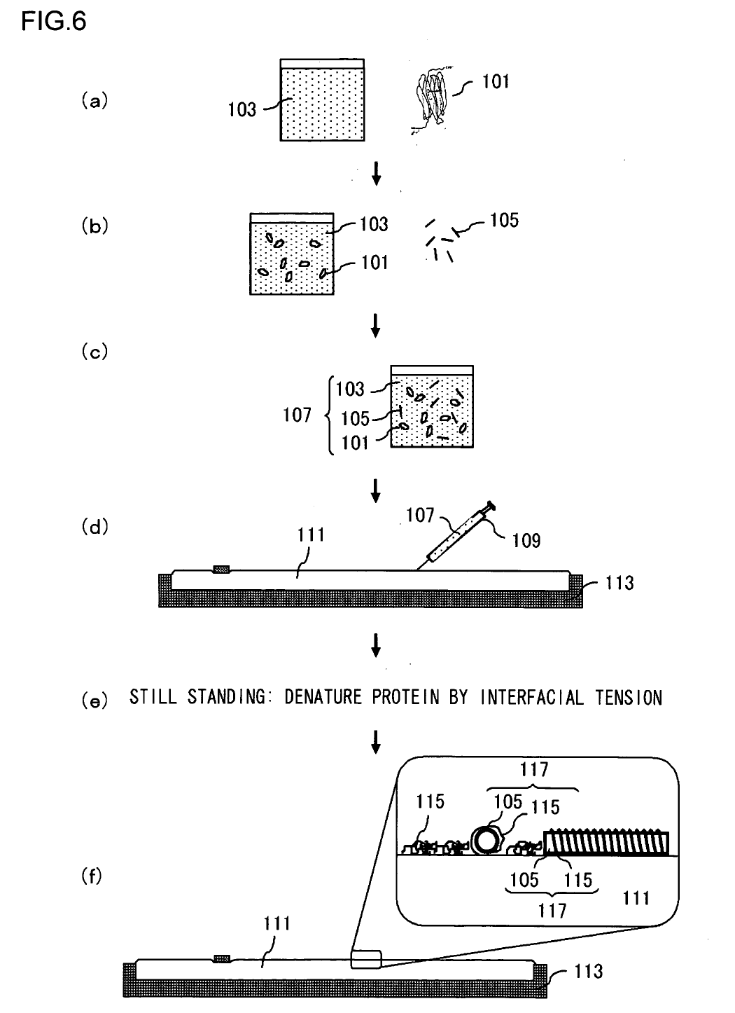

In this example, carbon nanotube structures 117 were prepared that consist of carbon nanotubes 105 and polypeptide chains of a denatured bacteriorhodopsin 115 wound about the carbon nanotubes 105. A production process of the carbon nanotube structures 117 is shown in FIG. 6.

First, purple membrane containing bacteriorhodopsin 101 was dispersed in a dispersion medium (FIG. 6(a)). The bacteriorhodopsin 101 for use in the present invention may be either purple membrane or bacteriorhodopsin present in purple membrane. In this example, purple membrane was used. Purple membranes can be isolated from halophiles, such as halobacterium salinarum. Purple membranes were isolated according to the method described in “Methods in Enzymology,” 31, A, pp. 667-678 (1974). A 33 v/v % aqueous solution of dimethylformamide (DMF) was used as the dispersion medium 103. Aside from the 33 v/v % aqueous solution of dimethylformamide (DMF), aqueous solutions of different organic solvents may be used as the dispersion medium 103.

An excess amount of the carbon nanotubes 105 was added to the dispersion solution of the bacteriorhodopsin 101 and the mixture was sonicated with a sonicator for more than 1 hour to disperse the components (FIG. 6(b)). Subsequently, remaining aggregates of the carbon nanotubes 105 were removed. The carbon nanotubes used were single-walled carbon nanotubes manufactured by CNI (Open end type, approx. 1 nm in diameter, purity=approx. 93%).

Using a syringe 109, the dispersion solution 107 (FIG. 6(c)) so obtained was gently spread over the surface of a subphase 111 placed in a reservoir (FIG. 6(d)). As a result, a monolayer of carbon nanotubes 105 was obtained. In this example, a Langmuir trough 113 was used as the reservoir and pure water having its pH adjusted to 3.5 with HCl was used as the subphase 111.

The monolayer of the carbon nanotubes 105 was allowed to stand to denature the bacteriorhodopsin 101 by the action of interfacial tension of the subphase 111. Since purple membranes are preferably left to stand at room temperature for at least 5 hours before bacteriorhodopsin in the purple membrane is denatured, the membranes were allowed to stand for 5 hours in this example (FIG. 6(e)). As a result, the denatured bacteriorhodopsin 115 became wound about the carbon nanotubes 105 (FIG. 6(f)).

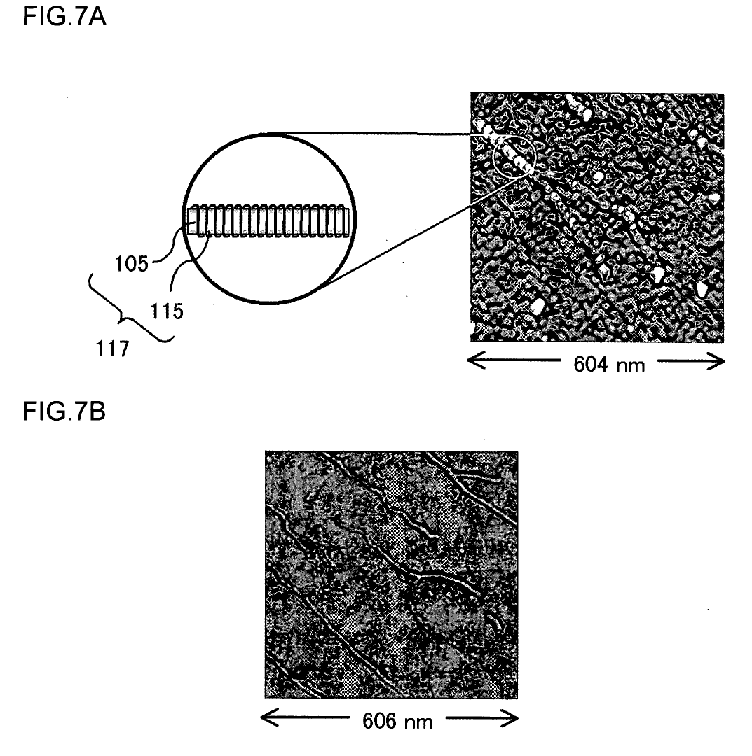

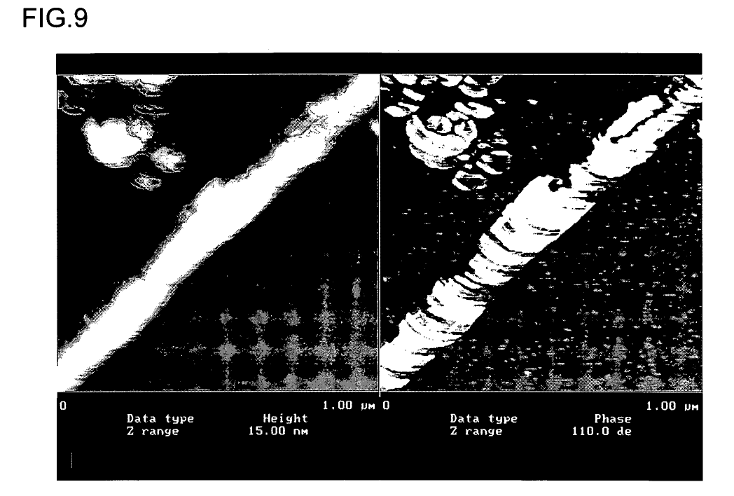

The resulting carbon nanotube structures 117 were observed with AFM and a transmission electron microscope (TEM). FIG. 7A and FIG. 9 each show an AFM image of the carbon nanotube structures 117. FIG. 7B is an AFM image of carbon nanotubes 105 obtained using a dispersion solution containing carbon nanotubes 105 but not bacteriorhodopsin 101. For AFM observation, a biological molecule visualization/measurement apparatus BMVM-X1 (NanoScope IIIa, Digital Instruments) was used. Silicon single crystal (NCH) was used as a probe. In FIGS. 7A and 7B, measurements were made using a tapping mode AFM with the observed range of 604 nm×604 nm (Z8 nm).

A comparison between FIG. 7A, FIG. 9 and FIG. 7B reveals that in FIG. 7A, the denatured bacteriorhodopsin 115 is wound about the carbon nanotube 105 with a specific pitch, forming a covering of the bacteriorhodopsin 115 over the surface of the carbon nanotube 105.

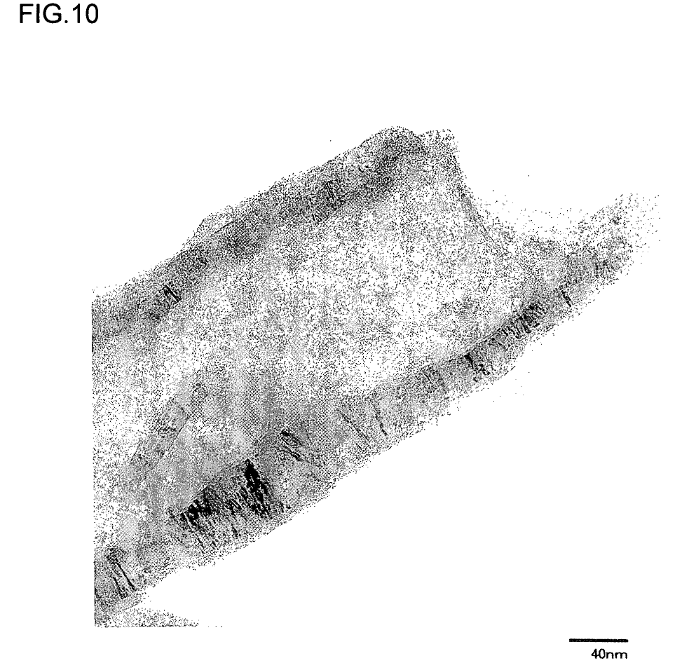

A further observation was made by TEM. Specifically, the carbon nanotube structures 117 formed over the liquid surface were transferred, along with the supporting monolayer, onto a TEM grid, which in turn was dried and was subjected to TEM. FIG. 10 is a TEM image of the carbon nanotube structure 117. As shown in FIG. 10, the denatured bacteriorhodopsin 115 was wound about the carbon nanotube 105 with a specific pitch, forming a dense wound layer over the surface of the carbon nanotube 105.

This example demonstrates successful preparation of the carbon nanotube structures 117 using a simple technique in which a dispersion solution containing bacteriorhodopsin 101 and carbon nanotubes 105 was spread over a liquid surface.

A four-month period cold storage test was conducted using the respective dispersion solutions used to form the spread films in FIGS. 7A and 7B. For the spread solution used in FIG. 7A, which was the dispersion solution containing the carbon nanotubes 105 and the bacteriorhodopsin 101, a 30-minute sonication recovered good dispersion of the components after the four-month storage period. In comparison, aggregated lumps of the carbon nanotubes 105 remained after a one-hour sonication in the spread solution used in FIG. 7B, which was the dispersion solution containing the carbon nanotubes 105 but not the bacteriorhodopsin 101, after the dispersion solution was stored for one month. Thus, the dispersibility was significantly reduced in the spread solution used in FIG. 7B.

It was thus demonstrated that bacteriorhodopsin 101, when added to the dispersion solution, serves to keep the carbon nanotubes 105 dispersed in the dispersion solution over an extended period of time and significantly increases redispersibility of the carbon nanotubes 105. Accordingly, a dispersion solution of the carbon nanotubes 105 has been obtained in which the carbon nanotubes can remain well-dispersed and that has high storage stability. Such a dispersion solution can serve as an intermediate in the production of the carbon nanotube structures 117.

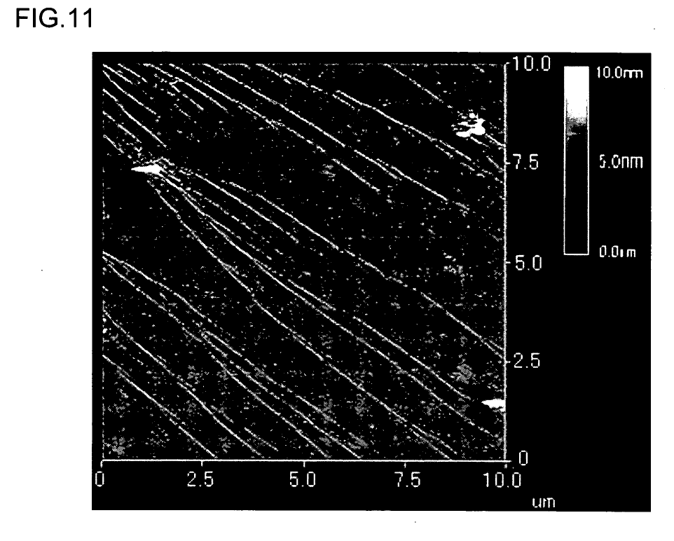

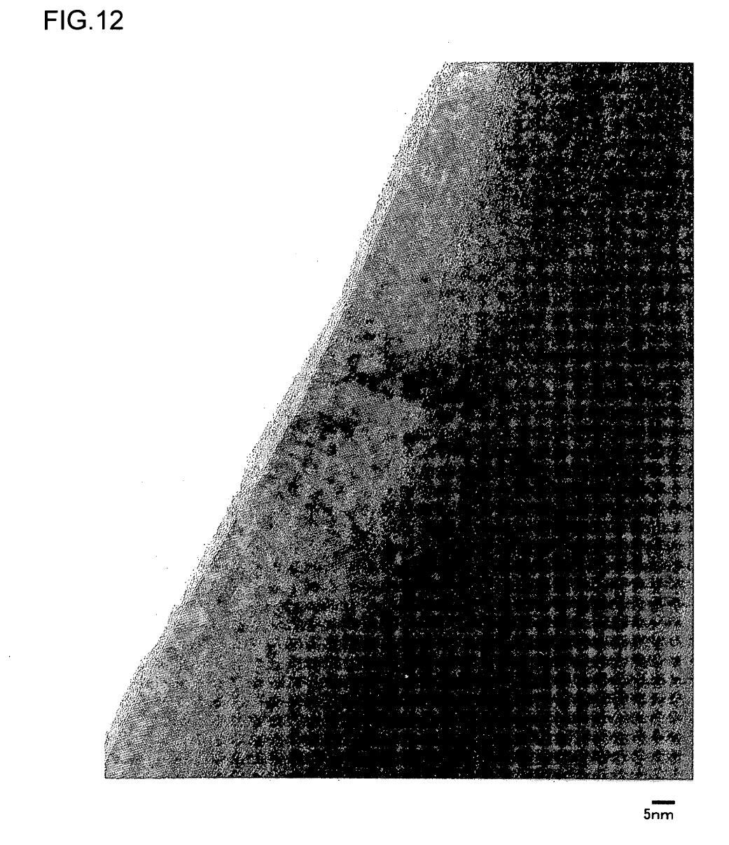

Using multi-walled carbon nanotubes manufactured by MTR Ltd. (Closed end type, a few tens to 200 nm in diameter, purity=approx. 95%) as the carbon nanotubes 105, carbon nanotube structures 117 were also prepared by the method described above with reference to FIG. 6. The resulting carbon nanotube structures 117 were observed with AFM and TEM. FIG. 11 is an AFM image of the carbon nanotube structure 117 prepared by using the multi-walled carbon nanotubes. FIG. 12 is a TEM image of the carbon nanotube structure 117 prepared by using the multi-walled carbon nanotubes.

As shown in FIG. 11, an oriented film in which the carbon nanotube structures 117 were oriented parallel to one another was obtained when multi-walled carbon nanotubes were used as the carbon nanotubes 105. A TEM observation revealed that a uniform layer of the denatured bacteriorhodopsin 115 was formed over the surface of the carbon nanotube 105, as shown in FIG. 12. Specifically, the carbon nanotube structures 117 formed over the liquid surface were transferred, along with the supporting monolayer, onto a TEM grid, which in turn was dried and was subjected to TEM. The layer of the denatured bacteriorhodopsin 115 was approximately 3 nm thick.

It has thus been demonstrated that carbon nanotube structures 131 that are suitable for stable production are obtained by using bacteriorhodopsin, a membrane protein. This example has also demonstrated stable production of the carbon nanotube structures 131 by using purple membranes in which bacteriorhodopsin molecules are embedded in an amphipathic lipid membrane.

An attempt was also made to prepare carbon nanotube structures 117 by using calf histone protein rather than the bacteriorhodopsin 101 to serve as the modified molecules 119. The histone protein, however, did not form a monolayer over the subphase 111, resulting in a failure to form the covering layer over the surface of the carbon nanotubes 105.

Another attempt was made to prepare carbon nanotube structures 117 by using the method described in the fourth embodiment above (FIGS. 8A through 8E), rather than the method described above with reference to FIG. 6. In this attempt, a 33 v/v % aqueous solution of dimethylformamide (DMF) was used as each of the dispersion medium 179 and the dispersion medium 181 shown in FIGS. 8A through 8E. Pure water having its pH adjusted to 3.5 with HCl was used as the subphase 125.

As a result, the carbon nanotube structures 117 in which the denatured bacteriorhodopsin 115 covered the surface of the carbon nanotubes 105 were obtained in a stable manner regardless of whether the carbon nanotubes 105 were single-walled or multi-walled. The resultant carbon nanotube structures 117 showed high dispersion stability.

When the order of spreading the dispersion solutions in the method shown in FIGS. 8A through 8E was switched around, that is, the dispersion solution 185 first spread over the subphase 125, followed by spreading of the dispersion medium 181, the carbon nanotube structures 117 were not successfully obtained.

Accordingly, it has been proven that by first placing carbon nanotubes 105 in the vicinity of the gas/liquid interface and then adding bacteriorhodopsin 101, the bacteriorhodopsin 101 can be denatured to expose its hydrophobic domains. This in turn brings about hydrophobic interactions between the bacteriorhodopsin and the carbon nanotubes 105, so that the bacteriorhodopsin covers the surface of the carbon nanotubes 105.

1. A carbon nanotube structure, comprising a carbon nanotube and a layer comprising a polymer that covers the sidewall of the carbon nanotube, the polymer being a membrane protein.

2. A method for solubilizing carbon nanotubes, comprising the step of adding carbon nanotubes to a dispersion medium containing a membrane protein.

3. A method for storing carbon nanotubes, comprising the step of maintaining carbon nanotubes in a liquid containing a membrane protein.

4. A carbon nanotube structure, comprising a carbon nanotube and a layer comprising a polymer that covers the sidewall of the carbon nanotube, the polymer being a water-insoluble biological polymer.

5. A carbon nanotube structure, comprising a carbon nanotube and a polymer that is wound about the carbon nanotube, the polymer being a membrane protein.

6. A carbon nanotube structure, comprising a carbon nanotube and a polymer that is wound about the carbon nanotube, the polymer being a water-insoluble biological polymer.

7. The carbon nanotube structure according to claim 4 or 6, wherein the water-insoluble biological polymer is a denatured protein.

8. A method for producing a carbon nanotube structure, comprising the steps of:
dispersing carbon nanotubes and a polymer in a dispersion medium to form a dispersion solution; and
spreading the dispersion solution over the surface of a liquid to cause the polymer to wind about the carbon nanotubes.

9. A method for producing a carbon nanotube structure, comprising the steps of:
dispersing carbon nanotubes and a polymer in a dispersion medium to form a dispersion solution; and
spreading the dispersion solution over the surface of a liquid to cause the polymer to cover the side wall of the carbon nanotubes.

10. The method according to claim 8, wherein the polymer is a protein, and the spreading of the dispersion solution over the liquid surface causes the protein to denature and the denatured protein is allowed to wind about the carbon nanotubes.

11. The method according to claim 9, wherein the polymer is a protein, and the spreading of the dispersion solution over the liquid surface causes the protein to denature and the denatured protein is allowed to cover the sidewall of the carbon nanotubes.

12. The method according to claim 10 or 11, wherein the protein is a membrane protein.
